(19) United States
(12) Patent Application Publication
(10) Pub. No.: US 2017/0042849 A1
(43) Pub. Date: Feb. 16, 2017

(54) COMPOSITION FOR BRAIN IMPROVEMENT

(71) Applicant: N.V. Nutricia, Zoetermeer, (NL)

(72) Inventors: Ruurd VAN ELBURG, Utrecht (NL); Nana BARTKE, Utrecht (NL); Johan GARSSEN, Utrecht (NL); Aletta Desiree KRANEVELD, Nigtevecht (NL)

(73) Assignee: N.V. Nutricia, Zoetermeer, (NL)

(21) Appl. No.: 15/304,279

(22) PCT Filed: Apr. 16, 2015
(86) PCT No.: PCT/NL2015/050255

§ 371 Date: Oct. 14, 2016

(30) Foreign Application Priority Data
Apr. 17, 2014 (NL) PCT/NL2014/050246

(51) Int. Cl. A61K 31/198; A61K 9/00; A61K 31/7076; A61K 31/7068; A61K 31/7072; A61K 31/7004; A61K 31/7016; A23L 33/175; A23L 33/12; A23L 33/17; A23L 33/115; A23L 33/135; A23L 33/00; A23L 33/125; A23L 33/21; A61K 31/202

(52) U.S. Cl. CPC A61K 31/198; A61K 31/202; A61K 9/0095; A61K 31/7076; A61K 31/7068; A61K 31/7072; A61K 31/7004; A61K 31/7016; A23L 33/175; A23L 33/12; A23L 33/17; A23L 33/115; A23L 33/135; A23L 33/40; A23L 33/125; A23L 33/21; A23V 2002/00; A23Y 2300/29

(57) ABSTRACT

The invention relates to the use of glutamine, preferably in the form of free amino acids, dipeptides and/or tripeptides, in the manufacture of a nutritional composition for improving exploratory behavior, memory performance, particularly spatial memory performance, and/or social interaction in an infant or toddler and/or treating or preventing impaired or decreased exploratory behavior, memory performance, particularly spatial memory performance, and/or social interaction in an infant or toddler.

### FIELD OF THE INVENTION

The present invention relates to nutritional compositions comprising glutamine for use in improving exploratory behavior, memory performance and/or social interaction. The invention is in particular suitable for infants with an age of 6 month or below, preterm infants, and for infants or young children suffering from inflammation, allergy or atopic disease.

### BACKGROUND OF THE INVENTION

Increasing attention is raised towards the connection between the gut and the brain and the role nutrition plays in this connection. A complex, bidirectional communication system exists between the gut and the brain, which ensures gastrointestinal homeostasis and digestion maintenance, and which in reverse direction may affect cognitive and psychological function and behavior. In some subjects this connection between the gut and brain is disturbed resulting in a co-morbidity between intestinal inflammation and increased anxiety, depression, altered pain sensitivity and/or decreased cognitive performance.

Especially in infants and toddlers such co-morbidity is unwanted since during infancy the cognitive system is still developing, and the disturbance of this development may have long lasting effects. Peri-natal brain plasticity increases the vulnerability to early stress conditions, such as repetitive pain, or inflammation, which may lead to abnormal cognitive development and behavior.

Breast-feeding is the preferred method of feeding infants. However, there are circumstances that make breast-feeding impossible or less desirable. In those cases infant formulae are a good alternative. The composition of modern infant formulae is adapted in such a way that it meets many of the special nutritional requirements of the fast growing and developing infant. Still further improvements can be made. The present invention relates to nutritional compositions for infants, in particular infant formulae, which comprises specific ingredients for improving exploratory behavior, memory performance and/or social interaction.

WO 20011/047107 and US 2011/086809 disclose a method for supporting retinal, intestinal and/or nervous system development in a neonate, providing arginine-glutamine dipeptide. Many, many further ingredients are listed as part of infant formulae which may be used to administer the dipeptide. The effects supported in the examples are the prevention of retinopathy of prematurity in a mouse model of oxygen-induced retinopathy, and the protection against intestinal and brain injury induced by hyperoxia in a mouse model monitoring caspase-3 activity and ‘intestinal damage score’.

Olubukunola et al. “Intestinal perforation in very low birth weight infants: growth and neurodevelopment at 1 year of age” J. Perinatology vol. 25, no. 9 (2005) 583-589 [XP002704986] compares growth and neurodevelopment in surviving very low birth weight infants with an intestinal perforation caused by necrotizing enterocolitis versus spontaneous intestinal perforation. Field et al. “A comparison of symptoms used by mothers and nurses to identify an infant with colic” Int. J. Nursing Studies vol. 31, no. 2 (1994) 201-215 [XP022870583] addresses ways in which parents believe to identify colic with a variety of factors, including eating behavior, maternal anxiety, baby's and mother's diets, and baby's stress. No interventions were disclosed.

US 2011/208153 describes formulations and methods for delivering water-soluble and lipid-soluble nutrients for preventing or correcting nutrient deficiencies to subjects requiring small-volume nutritional support, such as preterms. The nutritional formulation comprising fatty acids such as DHA and/or ARA, amino acids such as arginine and glutamine and other nutrients is suitable for delivery via nasogastric tube, intragastric feeding and transpyloric administration. Among many other components, prebiotics are merely mentioned as a further ingredient. B. breve is not mentioned among the various ingredients, and, according to its background description, glutamine may be an ingredient as a primary fuel for rapidly dividing cells, such as intestinal enterocytes and lymphocytes.

De Kieviet et al. “Effects of glutamine of brain development in very preterm children at school age” Pediatrics vol. 130, no. 5 (2012) 1121-1127 [SP009168039] studies the effect of short-term administration of glutamine to very preterm children in the first month after birth on brain development later in life, at school age. A similar study is reported in De Kieviet et al. “Effects of neonatal enteral glutamine supplementation on cognitive, motor and behavioral outcomes in very preterm and/or very low birth weight children at school age” British J. Nutrition vol 108, no. 12 (2012) 2215-2220. Based on the behavioral outcome tests used there, it was concluded that glutamine supplementation between day 3 and day 30 of life had neither beneficial nor detrimental effects on long-term cognitive, motor and behavioral outcomes of very preterm and/or VLBW children at school age, although visuomotor abilities were poorer in children that received glutamine.

### SUMMARY OF THE INVENTION

The inventors surprisingly found when employing animal models that dietary supplementation with glutamine resulted in improved exploratory behavior, memory performance and social interaction. In a preferred embodiment, particularly spatial memory was greatly and/or significantly improved. In another preferred embodiment, also social interaction was greatly and/or significantly improved. Direct, significant effects were observed immediate after intervention, not to be confused for any later-in-life effects.

The inventors used a cow's milk allergy (CMA) model which was associated with disturbed social interaction and impaired spatial memory, the mice previously sensitized to whey protein demonstrated less social interaction and lesser spatial memory compared to non-allergic control mice. For mice which had been supplemented with glutamine, social interaction and alternation or spatial memory levels were improved to levels comparable as for non-allergic mice. Also, data are herein also presented that interestingly show that nutritional supplementation of normal, non-allergic mice with a diet comprising glutamine leads to an improvement in exploratory behavior, memory performance, particularly spatial memory performance, and/or social interaction.

Based on these findings reported in the experimental part, in a first aspect the invention pertains to the use of glutamine for improving exploratory behavior, memory performance and/or social interaction and treating, preventing or reducing the risk of occurrence of decreased or impaired exploratory behavior, memory performance, particularly spatial memory performance, and/or social interaction in infants and young children, in particular infants with an age of 6 months or below, preterm infants, small for gestational age (SGA) infants, infants born via caesarian section, infants and young children suffering from or being at risk of allergy and infants and young children suffering from intestinal inflammation.

### LIST OF PREFERRED EMBODIMENTS

1. Use of glutamine in the manufacture of a nutritional composition for improving exploratory behavior, memory performance and/or social interaction in an infant or toddler and/or treating or preventing impaired or decreased exploratory behavior, memory performance and/or social interaction in an infant or toddler.

2. Use according to embodiment 1, wherein the nutritional composition further comprises either
a) one or more fatty acids selected from the group consisting of eicosapentaenoic acid, a C-10, a C-12, a C-14 and a C-16 fatty acid, or
b) one or more nucleotide(s) selected from the group consisting of cytidine 5′-monophosphate, uridine 5′-monophosphate, adenosine 5′-monophosphate, guanosine 5′-monophosphate and inosine 5′-monophosphate, or
c) a carbohydrate selected from the group consisting of glucose, maltose and sucrose, or a combination thereof,
or any combination of a), b) and c), and/or wherein the composition is substantially free of non-digestible oligosaccharides.

3. Use according to embodiment 1 or 2, wherein the infant or toddler is selected from the group consisting of infants with an age of 6 months or below, preterm infants, small for gestational age (SGA) infant, infants born via caesarian section, infants or toddlers suffering from allergy or infants or toddlers being at risk of allergy and infants or toddlers suffering from intestinal inflammation.

4. Use according to any one of the preceding embodiments, wherein memory performance is or comprises spatial memory performance or short-term or direct memory performance.

5. Use according to any one of the preceding embodiments, wherein the composition does not comprise the combination of Bifidobacterium breve and a non-digestible oligosaccharide.

6. Use according to any one of the preceding embodiments, wherein the C-10 fatty acid comprises capric acid, the C-12 fatty acid comprises lauric acid, the C14 fatty acid comprises myristic acid and the C-16 fatty acid comprises palmitic acid and/or beta-palmitate.

7. Use according to any one of the preceding embodiments, wherein the nutritional composition further comprises arachidonic acid and/or docosahexaenoic acid.

8. Use according to any one of the preceding embodiments, wherein the nutritional composition comprises protein, fat and/or digestible carbohydrates and is selected from the group consisting of an infant starter formula, an infant follow on formula, a toddler milk, a preterm formula, a post discharge formula and a human milk fortifier.

9. Use according to any one of the preceding embodiments, wherein glutamine is present in the form of free amino acids, dipeptides and/or tripeptides.

10. Use according to any one of the preceding embodiments, wherein the total amount of fatty acids of a) represents between 5 and 50% of total calories of the nutritional composition, preferably between 5 and 25%.

11. Use according to any one of the preceding embodiments, wherein the total amount of the nucleotides of b) is between 0.01 and 5 mg/100 kcal of the nutritional composition.

12. Use according to any one of the preceding embodiments, wherein the total amount of carbohydrates of c) represents between 5 and 50% of total calories of the nutritional composition, preferably between 5 and 25%.

13. Use of glutamine in the form of free amino acids and/or glutamine containing dipeptide and/or glutamine containing tripeptide in the manufacture of a nutritional composition for providing nutrition to infants or toddlers selected from the group consisting of infants with an age of 6 months or below, preterm infants, small for gestational age (SGA) infant, infants born via caesarian section, infants or toddlers suffering from allergy or infants or toddlers being at risk of allergy and infants or toddlers suffering from intestinal inflammation.

14. Use according to embodiment 13, said composition further comprising:
a) one or more of eicosapentaenoic acid, a C-10, a C-12, a C-14 or a C-16 fatty acid, or
b) one or more nucleotide(s) selected from the group of cytidine 5′-monophosphate, uridine 5′-monophosphate, adenosine 5′-monophosphate, guanosine 5′-monophosphate and inosine 5′-monophosphate, or
c) a carbohydrate selected from the group consisting of glucose, maltose and sucrose, or any combination of a), b) and c), and/or wherein the composition is substantially free of a non-digestible dietary fiber.

15. A method of improving exploratory behavior, memory performance and/or social interaction in an infant or toddler and/or treating, preventing or reducing the risk of occurrence of impaired or decreased exploratory behavior, memory performance, particularly spatial memory performance, and/or social interaction in an infant or toddler, comprising the enteral administration or oral ingestion of a composition comprising glutamine, preferably in the form of free amino acids, dipeptides and/or tripeptides.

16. The method according to embodiment 15, wherein the nutritional composition further comprises:
a) one or more of eicosapentaenoic acid, a C-10, a C-12, a C-14 or a C-16 fatty acid, or
b) one or more nucleotide(s) selected from the group of cytidine 5′-monophosphate, uridine 5′-monophosphate, adenosine 5′-monophosphate, guanosine 5′-monophosphate and inosine 5′-monophosphate, or
c) a carbohydrate selected from the group consisting of glucose, maltose and sucrose, or any combination of a), b) and c), and/or wherein the composition is substantially free of a non-digestible dietary fiber.

17. A nutritional composition comprising glutamine, preferably in the form of free amino acids, dipeptides and/or tripeptides for use in improving exploratory behavior, memory performance and/or social interaction in an infant or toddler and/or treating, preventing or reducing the risk of occurrence of impaired or decreased exploratory behavior, memory performance, particularly spatial memory performance, and/or social interaction in an infant or toddler.

18. The nutritional composition for use according to embodiment 17, further comprising:
a) one or more of eicosapentaenoic acid, a C-10, a C-12, a C-14 or a C-16 fatty acid, or
b) one or more nucleotide(s) selected from the group of cytidine 5′-monophosphate, uridine 5′-monophosphate, adenosine 5′-monophosphate, guanosine 5′-monophosphate and inosine 5′-monophosphate, or
c) a carbohydrate selected from the group consisting of glucose, maltose and sucrose, or any combination of a), b) and c), and/or wherein the composition is substantially free of a non-digestible dietary fiber.

19. A kit of parts comprising a first container comprising glutamine, preferably in the form of free amino acids, dipeptides and/or tripeptides, at least one non-digestible oligosaccharide selected from the group consisting fructo-oligosaccharides, galacto-oligosaccharides, gluco-oligosaccharides, arabino-oligosaccharides, mannan-oligosaccharides, xylo-oligosaccharides, fuco-oligosaccharides, arabinogalacto-oligosaccharides, glucomanno-oligosaccharides, galactomanno-oligosaccharides, sialic acid comprising oligosaccharides and uronic acid oligosaccharides and a carbohydrate selected from the group consisting of glucose, maltose, sucrose and maltodextrin, or a combination thereof; and a second container comprising Bifidobacterium breve.

20. Kit of parts according to embodiment 19, wherein glutamine is present in the first container in an amount of between 1.5 and 20 wt. %, preferably between 1.5 and 10 wt %, the non-digestible oligosaccharide is preferably present in an amount of between 0.5 to 20 wt. %, and the carbohydrate is preferably present in an amount of at least 50 wt. %, more preferably at least 60 wt. %., even more preferably at least 70 wt. %, most preferably at least 80 wt. %.

21. Kit of parts according to embodiment 19 or 20, wherein fructo-oligosaccharides and galacto-oligosaccharides are present as non-digestible oligosaccharides, in a weight ratio of glutamine to the sum of fructo-oligosaccharides and galacto-oligosaccharides of between 2:1 and 1:4, more preferably between 1:1 and 1:3, most preferably around about 1:2.

### DETAILED DESCRIPTION OF THE INVENTION

The present invention thus concerns a method for improving exploratory behavior, memory performance, particularly spatial memory performance, and/or social interaction in an infant or toddler; and/or treating, preventing or reducing the risk of occurrence of decreased or impaired exploratory behavior, memory performance, particularly spatial memory performance, and/or social interaction in an infant or toddler, comprising administering to the infant or toddler a nutritional composition comprising glutamine, preferably in the form of free amino acids, dipeptides and/or tripeptides. The invention can also be worded as the use of glutamine, preferably in the form of free amino acids, dipeptides and/or tripeptides, for the manufacture of a nutritional composition for improving exploratory behavior, memory performance, particularly spatial memory performance, and/or social interaction in an infant or toddler; and/or treating or preventing impaired or decreased exploratory behavior, memory performance, particularly spatial memory performance, and/or social interaction in an infant or toddler. The invention can also be worded as a nutritional composition comprising glutamine, preferably in the form of free amino acids, dipeptides and/or tripeptides, for use in improving exploratory behavior, memory performance, particularly spatial memory performance, and/or social interaction in an infant or toddler; and/or for use in treating or preventing impaired or decreased exploratory behavior, memory performance, particularly spatial memory performance, and/or social interaction in an infant or toddler.

In a preferred embodiment of the present method or use, the infant or toddler is selected from the group consisting of infants with an age of 6 months or below, preterm infants, small for gestational age (SGA) infant, infants born via caesarian section, infants or toddlers suffering from allergy or infants or toddlers being at risk of allergy and infants or toddlers suffering from intestinal inflammation. In a preferred embodiment of the present method or use is for providing nutrition to the infant or toddler.

In a preferred embodiment, the above method or use pertains to providing nutrition to the infant or toddler selected from the group consisting of infants with an age of 6 months or below, preterm infants, small for gestational age (SGA) infant, infants born via caesarian section, infants or toddlers suffering from allergy or infants or toddlers being at risk of allergy and infants or toddlers suffering from intestinal inflammation.

The invention also concerns a method for feeding or a method of providing nutrition to an infant or toddler selected from the group consisting of infants with an age of 6 months or below, preterm infants, small for gestational age (SGA) infant, infants born via caesarian section, infants or toddlers suffering from allergy or infants or toddlers being at risk of allergy and infants or toddlers suffering from intestinal inflammation comprising administering a nutritional composition comprising glutamine, preferably in the form of free amino acids, dipeptides and/or tripeptides. In other words the invention concerns the use of a nutritional composition comprising glutamine, preferably in the form of free amino acids, dipeptides for providing nutrition to an to an infant or toddler selected from the group consisting of infants with an age of 6 months or below, preterm infants, small for gestational age (SGA) infant, infants born via caesarian section, infants or toddlers suffering from allergy or infants or toddlers being at risk of allergy and infants or toddlers suffering from intestinal inflammation. The invention can also be worded as a nutritional composition comprising glutamine, preferably in the form of free amino acids, dipeptides and/or tripeptides for use in providing nutrition to an to an infant or toddler selected from the group consisting of infants with an age of 6 months or below, preterm infants, small for gestational age (SGA) infant, infants born via caesarian section, infants or toddlers suffering from allergy or infants or toddlers being at risk of allergy and infants or toddlers suffering from intestinal inflammation.

### Glutamine

The nutritional composition of the present invention comprises glutamine. Throughout this description this is also referred to as nutritional composition comprising glutamine or glutamine enriched nutritional composition. Glutamine in the present invention refers to L-glutamine. Glutamine is one of the most abundant amino acids in plasma and human milk and is considered conditionally essential in preterm infants. Glutamine is utilized as a source of energy and for nucleotide synthesis in all rapidly dividing cells, such as the intestinal lining and certain immune cells. In the brain, glutamine is a substrate for neurotransmitters and an important source of energy for the nervous system.

Infants with an age of 6 months or below, preterm infants, small for gestational age (SGA) infant, infants born via caesarian section, infants or toddlers suffering from allergy or infants or toddlers being at risk of allergy and infants or toddlers suffering from intestinal inflammation may be especially susceptible to glutamine depletion as nutritional supply of glutamine is limited in the first weeks after birth. Furthermore the endogenous capacity to synthesize glutamine from glutamate may be compromised in these infants and in particular is not fully developed in preterm and SGA infants.

Glutamine is preferably present in an easily absorbable form. Because of the immaturity of the intestinal tract of the SGA and/or preterm infant, glutamine present in intact protein is less easily absorbed. Therefore the glutamine is preferably present in the form of free amino acids and/or di- and tripeptides comprising glutamine, most preferably glutamine comprising dipeptide and/or free glutamine. Free glutamine and glutamine comprising dipeptide and glutamine comprising tripeptide are commercially available, for example at Ajinomoto, USA.

In one embodiment, the nutritional composition of the present invention comprises less than 0.3 g/l arginine-glutamine dipeptide, when the composition is in liquid form, or less than 2.5 wt. %, preferably less than 1.5 wt. %, more preferable less than 1 wt. %, even more preferably less than 0.5 wt. % arginine-glutamine dipeptide based on dry weight of the composition. In one embodiment, the nutritional composition of the present invention does not comprise arginine-glutamine dipeptide.

The nutritional composition of the present invention preferably comprises glutamine levels higher then normally present in human milk protein or standard preterm formula based on cow's milk derived protein. Preferably the nutritional composition of the present invention comprises at least 12 wt. %, more preferably at least 15 wt. %, even more preferably at least 30 wt. % glutamine based on total protein. Preferably the nutritional composition of the present invention comprises at least 1.5 wt. %, more preferably at least 2 wt. %, even more preferably at least 4 wt. % and preferably up to 10 wt. %, more preferably up to 20 wt % glutamine based on dry weight of the nutritional composition. Preferably the nutritional composition of the present invention comprises at least 0.3 g, more preferably at least 0.5 g, even more preferably at least 1 g glutamine based on 100 kcal. Preferably the composition of the present invention comprises at least 0.4 g, more preferably at least 0.6 g, even more preferably at least 1.25 g glutamine per 100 ml. Preferably the nutritional composition of the present invention comprises at least 12 wt. %, more preferably at least 15 wt. %, even more preferably at least 30 wt. % glutamine in the form of free amino acid and if present glutamine containing dipeptide and glutamine containing tripeptide based on total protein. Preferably the nutritional composition of the present invention comprises at least 1.5 wt. %, more preferably at least 2 wt. %, even more preferably at least 4 wt. % and preferably up to 10 wt. %, more preferably up to 20 wt % glutamine in the form of free amino acid, and if present, glutamine containing dipeptide and glutamine containing tripeptide, based on dry weight of the nutritional composition. Preferably the nutritional composition of the present invention comprises at least 0.3 g, more preferably at least 0.5 g, even more preferably at least 1 g glutamine in the form of free amino acid, and if present, glutamine containing dipeptide and glutamine containing tripeptide, based on 100 kcal.

Preferably the present nutritional composition comprising glutamine is in dry form, preferably a powder. This powder is suitable for reconstitution with water or another aqueous phase. When glutamine is in powder form it advantageously has a better shelf life. Glutamine, in particular free glutamine and glutamine dipeptide, is more stable when stored in dry form.

In the aforementioned method or use according to the invention, the nutritional composition comprising glutamine preferably further comprises:

a) one or more fatty acids selected from the group consisting of eicosapentaenoic acid, a C-10, a C-12, a C-14 and a C-16 fatty acid, or
b) one or more nucleotide(s) selected from the group consisting of cytidine 5′-monophosphate, uridine 5′-monophosphate, adenosine 5′-monophosphate, guanosine 5′-monophosphate and inosine 5′-monophosphate, or
c) a carbohydrate selected from the group consisting of glucose, maltose and sucrose, or a combination,
or any combination of a), b) and c),
and/or (d) the composition is substantially free of non-digestible oligosaccharides.

In one embodiment, the composition preferably comprises one or more nucleotide(s) selected from the group consisting of cytidine 5′-monophosphate, uridine 5′-monophosphate, adenosine 5′-monophosphate, guanosine 5′-monophosphate and inosine 5′-monophosphate, most preferably at least cytidine 5′-monophosphate, uridine 5′-monophosphate and adenosine 5′-monophosphate. The sum of the nucleotide(s) optionally present in the composition preferably amounts to 0.1-10 mg per 100 kcal of the composition, more preferably 0.5 to 5 mg per 100 kcal of the composition.

In one embodiment, the composition preferably comprises one or more fatty acids selected from the group consisting of eicosapentaenoic acid, a C-10, a C-12, a C-14 and a C-16 fatty acid. The composition preferably comprises eicosapentaenoic acid (EPA), capric, lauric, myristic and/or palmitic fatty acid, preferably at least EPA and/or palmitic fatty acids.

In one embodiment, the composition preferably comprises glucose, maltose and/or sucrose.

In one embodiment, the composition comprises any combination of a), b) and c), most preferably a), b) and c).

In one embodiment, the composition does not comprise the combination of Bifidobacterium breve and a non-digestible oligosaccharide, preferably no B. breve at all.

### Compositions

The present invention advantageously concerns a composition wherein the fatty acids of a) provide 5 to 50% of the total calories, the protein provides 5 to 50% of the total calories, and the carbohydrates of c) provide 1 to 50% of the total calories. Preferably, in the present composition the fatty acids of a) provide 5 to 25% of the total calories, the protein provides 7.5 to 12.5% of the total calories, and the carbohydrate provides 5 to 25% of the total calories. For calculation of the % of total calories for the protein component, the total of energy provided by the proteins, peptides and amino acids needs to be taken into account.

The present composition preferably comprises at least one lipid comprising the fatty acids of a) selected from the group consisting of animal lipid (excluding human lipids) and vegetable lipids. Preferably the present composition comprises a combination of vegetable lipids and at least one oil selected from the group consisting of fish oil, animal oil, algae oil, fungal oil, and bacterial oil.

The present composition excludes human milk.

The present composition preferably comprises protein. The protein component used in the nutritional preparation is preferably selected from the group consisting of non-human animal proteins (preferably milk proteins, preferably proteins from cow's milk), vegetable proteins (preferably soy protein and/or rice protein), free amino acids and mixtures thereof. The present composition preferably contains casein, whey, hydrolyzed casein and/or hydrolyzed whey protein. Preferably the protein comprises intact proteins, more preferably intact bovine whey proteins and/or intact bovine casein proteins. As the present composition is preferably suitably for use by infants suffering from or at risk of allergy, the protein is preferably selected from the group consisting of hydrolyzed milk protein, more preferably hydrolyzed whey protein. As the present composition is preferably suitably for use by preterm or SGA infants, the protein is preferably selected from the group consisting of hydrolyzed milk protein, more preferably selected from the group consisting of hydrolyzed whey protein and hydrolyzed casein.

The present composition may further comprise at least 35 wt. %, more preferably at least 50 wt. %, more preferably at least 75 wt. %, even more preferably at least 90 wt. %, most preferably at least 95 wt. % lactose. The present composition preferably comprises at least 25 grams lactose per 100 gram dry weight of the present composition, preferably at least 40 grams lactose/100 gram.

The liquid nutritional composition preferably has a caloric density between 0.1 and 2.5 kcal/ml, even more preferably a caloric density of between 0.5 and 1.5 kcal/ml, most preferably between 0.6 and 0.8 kcal/ml. The amount of nutritional composition administered per day is preferably between 50 and 2000 ml, more preferably between 200 and 1500, most preferably between 400 and 1000 ml.

In one embodiment the nutritional composition of the present invention is a preterm formula. The preterm formula comprises all macro- and micronutrients needed for preterm or SGA infants so as to achieve a growth similar to fetal growth coupled with satisfactory functional development.

In a preferred embodiment the preterm formula comprises from 5 to 25 wt. % protein, preferably 9 to 20 wt. %, more preferably 13 to 18 wt. % protein based on the dry weight of the preterm formula. In a preferred embodiment the preterm formula comprises from 1.8 to 3.0 g protein, preferably, preferably 2.0 to 3.0 g, preferably 2.5 g to 2.6 g protein, per 100 ml.

Preferably the preterm formula of the present invention comprises at least 12 wt. %, more preferably at least 15 wt. %, even more preferably at least 30 wt. % glutamine based on total protein. Preferably the preterm formula of the present invention comprises no more than 80 wt. %, more preferably no more than 50 wt. % glutamine based on total protein. Preferably the preterm formula of the present invention comprises at least 1.5 wt. %, more preferably at least 2 wt. %, even more preferably at least 4 wt. % glutamine based on dry weight of the preterm formula. Preferably the preterm formula of the present invention comprises no more than 20 wt. %, more preferably no more than 10 wt. %, more preferably up to 20 wt % glutamine based on dry weight of the preterm formula. Preferably the nutritional composition of the present invention comprises at least 0.3 g, more preferably at least 0.5 g, even more preferably at least 1 g glutamine based on 100 kcal of the preterm formula. Preferably the preterm formula of the present invention comprises no more than 5 g, even more preferably no more than 2 g glutamine based on 100 kcal of the preterm formula.

The preterm formula of the present invention in ready to drink form has in a preferred embodiment about 70 to 90 kcal, preferably 75 to 85 kcal per 100 ml.

Preferably the preterm formula has an osmolarity below 450 mOsmol/l, more preferably below 400, even more preferably below 350. Particularly in preterm infants, a too high osmolarity is a disadvantage.

In one embodiment the present invention concerns a supplement, suitable to fortify human milk, to fortify human milk fortified with a standard human milk fortifier or to fortify a standard preterm formula. In the context of this invention, a supplement does not comprise all macro- and micronutrients needed for preterm infants so as to achieve a growth similar to fetal growth coupled with satisfactory functional development.

In one embodiment the nutritional composition of the present invention is a post discharge formula. The post discharge formula comprises all macro- and micronutrients needed for preterm infants so as to achieve a growth similar to fetal growth coupled with satisfactory functional development.

Thus in one embodiment the nutritional composition according to the present invention or for use according to the present invention comprises protein, fat and/or digestible carbohydrates and is selected from the group consisting of an infant starter formula, an infant follow on formula, a toddler milk, a preterm formula, a post discharge formula and a human milk fortifier.

In one embodiment, the present composition does not comprise Bifidobacterium breve. Bifidobacterium breve is a Gram-positive, anaerobic, branched rod-shaped bacterium. However, if the composition comprises B. breve, it may have at least 95% identity of the 16 S rRNA sequence when compared to the type strain of B. breve ATCC 15700, more preferably at least 97% identity (Stackebrandt & Goebel, 1994, Int. J. Syst. Bacteriol. 44:846-849). Examples of B. breve strains are those isolated from the faeces of healthy human milk-fed infants. Typically, these are commercially available from producers of lactic acid bacteria, but they can also be directly isolated from faeces, identified, characterized and produced. The present composition may contain at least one B. breve selected from the group consisting of B. breve M-16V and B. breve CNCM I-2219, most preferably M-16V. B. breve 1-2219 was published in WO 2004/093899 and was deposited at the Collection Nationale de Cultures de Microorganisms, Institute Pasteur, Paris, France on 31 May 1999 by Compagnie Gervais Danone. B. breve M-16V was deposited as BCCM/LMG23729 and is commercially available from Morinaga. Milk Industry Co., Ltd.

If the composition comprises B. breve, the composition preferably contains 10² to 10¹³ colony forming units (cfu) B. breve per gram dry weight of the present composition, preferably 10⁴ to 10¹², more preferably 10⁵ to 10¹⁰, most preferably from 10⁵ to 1×10⁸ cfu B. breve per gram dry weight of the present composition. The dose of B. breve according to the present invention is preferably administered at a daily dose of 10² to 10¹³, more preferably from 10⁵ to 10¹², most preferably from 10⁷ to 5×10⁹ colony forming units (cfu). Preferably the composition comprises 10³ to 10¹³ cfu B. breve per 100 ml, more preferably 10⁶ to 10¹¹ cfu B. breve per 100 ml, most preferably 10⁷ to 10⁹ cfu B. breve per 100 ml.

In one embodiment, the composition may be free from non-digestible oligosaccharides (NDO).

The term “oligosaccharide” as used in the present invention preferably refers to a saccharide with a degree of polymerization (DP) of 2 to 250, preferably a DP of 2 to 100, more preferably of 2 to 60. However, if the present composition comprises non-digestible oligosaccharides it is understood that in the context of this invention a saccharide with a DP in a certain range may include a mixture of saccharides with different average DP's, for example, if an oligosaccharide with a DP of 2 to 100 is included in the present composition, this may include compositions which contain oligosaccharides with an average DP between 2 and 5, an average DP between 50 and 70 and an average DP between 7 and 60. The term “non-digestible oligosaccharide” as used in the present invention refers to oligosaccharides which are not or only partially digested in the intestine by the action of acids or digestive enzymes present in the human upper digestive tract (small intestine and stomach) but which are fermented by the human intestinal flora.

The non-digestible oligosaccharide may be selected from the group consisting of fructo-oligosaccharides (such as inulin), galacto-oligosaccharides (such as transgalacto-oligosaccharides or beta-galacto-oligosaccharides), gluco-oligosaccharides (such as gentio-, nigero- and cyclodextrin-oligosaccharides), arabino-oligosaccharides, mannan-oligosaccharides, xylo-oligosaccharides, fuco-oligosaccharides, arabinogalacto-oligosaccharides, glucomanno-oligosaccharides, galactomanno-oligosaccharides, sialic acid comprising oligosaccharides and uronic acid oligosaccharides. In one embodiment, the present nutritional composition comprises of 0.5 to 20 wt. % non-digestible oligosaccharides per g dry weight of the nutritional composition. Preferably the present composition comprises fructo-oligosaccharides and/or galacto-oligosaccharides, more preferably galacto-oligosaccharides, most preferably transgalacto-oligosaccharides. In a preferred embodiment the composition comprises a mixture of transgalacto-oligosaccharides and fructo-oligosaccharides. Preferably the present composition comprises galacto-oligosaccharides with a DP of 2-10, preferably with an average DP between 2 and 10, and/or fructo-oligosaccharides with a DP of 2-60, preferably with an average DP between 2 and 60, preferably with an average DP between 10 and 60, preferably with an average DP between 20 and 60. Preferably the composition comprises galacto-oligosaccharides and fructo-oligosaccharides in a weight ratio of 20 to 0.5, more preferably 20 to 1, most preferably from 12 to 2. Transgalacto-oligosaccharides (TOS) are for example sold under the trademark Vivinal™ (Borculo Domo Ingredients, Netherlands). Other terms for fructooligosaccharides include inulin, fructopolysaccharide, polyfructose, fructans and oligofructose. Fructo-oligosaccharides may be used as inulin, for example available under the tradename “Raftilin HP®”, (Orafti).

In one embodiment, the composition comprises of 80 mg to 2 g non-digestible oligosaccharides per 100 ml, more preferably 150 mg to 1.50 g, even more preferably 300 mg to 1 g per 100 ml. Based on dry weight, the composition preferably comprises 0.25 wt. % to 20 wt. %, more preferably 0.5 wt. % to 10 wt. %, even more preferably 1.5 wt. % to 7.5 wt. % non-digestible oligosaccharides. The composition may comprise 10² to 10¹³ cfu B. breve per gram and 0.25 wt. % to 20 wt. % non-digestible oligosaccharides based on dry weight, more preferably 10⁵ to 10¹⁰ cfu B. breve per gram and 0.5 wt. % to 10 wt. % non-digestible oligosaccharides based on dry weight. In one embodiment, the composition comprises 10³ to 10¹³ cfu B. breve and 80 mg to 2 g non-digestible oligosaccharides per 100 ml, more preferably 10⁶ to 10¹¹ cfu B. breve and 300 mg to 1 g non-digestible oligosaccharides per 100 ml. The non-digestible oligosaccharides could involve the mixture of galacto-oligosaccharides and fructo-oligosaccharides as defined above.

In one embodiment, the present composition comprises long chain poly unsaturated fatty acids (LC-PUFA), more preferably n-3 and n-6 LC-PUFA, even more preferable arachidonic acid (ARA) and docosahexaenoic acid (DHA). The DHA content preferably does not exceed 5 wt. %, more preferably does not exceed 1 wt. %, but is preferably at least 0.1 wt. % of the total fatty acid. The present composition preferably comprises relatively low amounts of ARA. The n-6 LC-PUFA content preferably does not exceed 5 wt. %, more preferably does not exceed 0.8 wt. %, more preferably does not exceed 0.75 wt. %, even more preferably does not exceed 0.5 wt. % based on total fatty acids. The weight ratio n-6 LC-PUFA/n-3 LC-PUFA, in particular the weight ratio of ARA/DHA in the present infant nutrition is preferably from 3 to 0.5, more preferably from 2 to 1. Preferably the weight ratio is above 1. These ratio's ensure an optimal brain functioning.

In a particular embodiment, the present invention relates to a kit-of-parts suitable and intended for feeding preterm infants comprising two different containers each with different contents and instructions for use. Said kit of parts comprises a first container comprising glutamine, preferably in the form of free amino acids, dipeptides and/or tripeptides, at least one non-digestible oligosaccharide selected from the group consisting of fructo-oligosaccharides, galacto-oligosaccharides, gluco-oligosaccharides, arabino-oligosaccharides, mannan-oligosaccharides, xylo-oligosaccharides, fuco-oligosaccharides, arabinogalacto-oligosaccharides, glucomanno-oligosaccharides, galactomanno-oligosaccharides, sialic acid comprising oligosaccharides and uronic acid oligosaccharides, more preferably a combination of at least fructo-oligosaccharide (FOS) and galacto-oligosaccharides (GOS), and a carbohydrate selected from the group consisting of glucose, maltose, sucrose and maltodextrin, or a combination; and a second container comprising Bifidobacterium breve.

In a preferred embodiment, the first container of the kit of parts comprises glutamine in free amino acid form, as a glutamine dipeptide, as a glutamine tri-peptide and/or an arginine-glutamine dipeptide, with fructo-oligosaccharide (FOS or lcFOS) and galacto-oligosaccharides (GOS or ‘short chain’ (sc) GOS having a DP of 2-10) present as well. GOS and FOS could be used as described elsewhere in the specification. The weight ratio of glutamine to the sum of non-digestible oligosaccharides (sc)GOS and (long chain′ (lc) [DP 10-60])FOS preferably lies between 2:1 and 1:4, more preferably lies between 1:1 and 1:3, most preferably lies around 1:2.

Preferably, glutamine is present in the first container in an amount of at least 1.5 wt. %, more preferably at least 2 wt. %, even more preferably at least 4 wt. % and preferably up to 10 wt. %, more preferably up to 20 wt % glutamine on dry weight basis.

Preferably, the non-digestible oligosaccharide(s) is/are present in the first container in an amount of between 0.5 to 20 wt. % on dry weight basis, more preferably 1.0 wt. % to 15 wt. %, even more preferably 5.0 wt. % to 10 wt. %.

Preferably, the carbohydrate selected from the group consisting of glucose, maltose, sucrose and maltodextrin or any combination thereof is present in the first container in an amount of at least 50 wt. %, more preferably at least 60 wt. %., even more preferably at least 70 wt. %, most preferably at least 80 wt. %. Most preferably, the carbohydrate is maltodextrin, preferably present in an amount of at least 50 wt. %, more preferably at least 60 wt. %., even more preferably at least 70 wt. %, most preferably at least 80 wt. %.

Preferably said container is a sachet (or a bundle with from 2 to 100 individual sachets) or a tin can. Preferably, said glutamine/GOS/FOS is present in dry form, preferably in powdered form. In one embodiment, said first container contains glutamine/GOS/FOS in sterilized liquid form, more preferably in a concentrated form or as a daily unit dose to prevent spoilage.

In a preferred embodiment, the kit of parts comprises a container which comprises Bifidobacterium breve M16V, preferably as described above under the header “Bifidobacterium breve”. Preferably said container is a sachet, or a bundle with from 2 to 100 individual sachets. Preferably, said B. breve is present in dry form, preferably in powdered form. Said container preferably comprises B. breve as a daily unit dose to prevent spoilage.

The optional instructions for use of the kit of parts preferably contain instructions to use glutamine, GOS/FOS and B. breve expressed either in g/kg/day or g/100 mL ready-to-feed product, such as:

a) L-glutamine from 0.05 to 0.5 g/100 mL, preferably between 0.1 and 0.3 g/100 mL and GOS/FOS (preferably in a 9:1 weight ratio) from 0.1 to 1.0 g/100 mL, preferably from 0.2 to 0.6 g/100 mL, and/or,
b) L-glutamine to be provided in a dose of 0.1 to 0.6 g/kg/day, preferably to be provided in a dose of 0.2 to 0.4 g/kg/day and GOS/FOS (preferably in a 9:1 weight ratio) to be provided in a dose of 0.2 to 1.2 g/kg/day, preferably to be provided in a dose of 0.4 to 0.8 g/kg/day, and
c) B. breve to be provided in a daily dose of between 0.5*10⁹ cfu to 1*10¹⁰, preferably to be provided in a daily dose of between 1*10⁹ cfu to 5*10⁹ cfu. The daily dose is to be divided equally over all individual feeding dosages given over the entire day, but preferably as a single dose. More specifically, B. breve is to be provided in a daily dose of 3×10⁹ cfu per day for infants with birth weight ≧1000 and 1.5×10⁹ cfu per day for preterm infants with birth weight <1000 g until an enteral feed of 50-60 ml/kg/day is reached, after which a dose of 3×10⁹ cfu is to be provided per day.

The contents of the kit of parts are intended to be added to human milk or infant formula to obtain the ready-to-feed product.

### Application

The present method or use is specifically intended for infants and/or toddlers. Infants have an age of 0-12 months, toddlers have an age of 12-36 months.

The present composition is preferably enterally administered, more preferably orally. The present composition is preferably a nutritional formula, preferably an infant formula. The present composition can advantageously be applied as a complete nutrition for infants. The present composition preferably comprises lipid, protein, and carbohydrate and is preferably administered in liquid form. The present invention includes dry compositions, e.g. powders, which are accompanied with instructions as to mix said dry compositions, in particular nutritional formula, with a suitable liquid, e.g. water.

The present invention preferably relates to a method for feeding an infant selected from the group consisting of preterm (=premature) infants and very premature infants. Preterm infants are born before the end of the 37th week of pregnancy. Very preterm infants are born before the end of the 32th week of pregnancy. SGA infants are those whose birth weight lies below the 10th percentile for that gestational age. They have usually been the subject of intrauterine growth restriction (IUGR). Premature and/or SGA infants include low birth weight infants (LBW infants), very low birth weight infants (VLBW infants), and extremely low birth weight infants (ELBW infants). LBW infants are defined as infants with a weight less than 2500 g. VLBW infants as infants with a weight which is less than 1500 g, and ELBW infants as infants with a weight less than 1000 g.

The present inventions concerns improving exploratory behavior, memory performance (preferably spatial memory performance) and/or social interaction in an infant or toddler. As evidenced in the examples, the above effects are observed directly, i.e. immediate upon administration, preferably within 24 months, more preferably within 12 months, more preferably with 6 months, most preferably within 3 months from the start of intervention. Memory performance thus preferably is or comprises spatial memory performance or ‘short-term’ or direct memory performance, not to be mistaken for any later in life effects that may arise only after at least 36 months or at least 12 months after intervention. Memory performance preferably is or comprises spatial memory performance.

Also the present invention concerns treating or preventing (or reducing the risk of) decreased exploratory behavior, memory performance (preferably spatial memory performance) and/or social interaction in an infant or toddler selected from the group consisting of infants or toddlers suffering from allergy, infants or toddlers being at risk of allergy, infants or toddlers suffering from intestinal inflammation, and preterm infants. In a particularly preferred embodiment, the invention concerns treating or preventing (or reducing the risk of) exploratory behavior, memory performance (preferably spatial memory performance) and/or social interaction in an infant or toddler selected from the group consisting of infants or toddlers suffering from allergy, infants or toddlers being at risk of allergy, infants or toddlers suffering from intestinal inflammation, and preterm infants.

Hence the present invention provides a method for providing nutrition to a human infant and/or toddler, said method comprising administering to the infant and/or toddler the present composition. Preferably the infant and/or toddler has an age between 0 and 36 month, more preferably between 0 and 18 month, even more preferably between 0 and 12 months, most preferably between) and 6 months.

### EXAMPLES

### Example 1

### Dietary Intervention with Glutamine Improves Social Interaction, Cognitive Performance and Behavioural Performance in a Cow's Milk Allergy Model in Mice

Pathogen-free, male C3H/HeOuJ mice (4 weeks old, Charles River Laboratories) mice were bred and raised on a cow's milk protein-free diet (Special Diet Services, Witham, UK) and housed on a 12 h light-dark cycle with access to food and water ad libitum. All animal procedures were conducted in accordance with the guidelines of the Dutch Committee of Animal Experiments. The mice were sensitized orally with 0.5 mL homogenized whey protein (40 mg/mL PBS) and cholera toxin (CT, 20 μg/mL PBS, Quadratech Diagnostics) as an adjuvant; sham-sensitized mice received CT alone. Mice were orally boosted once a week for 5 consecutive weeks. One week after the last sensitization, mice were challenged orally with 0.5 mL whey (100 mg/mL PBS).

The mice were fed a control diet, or a diet supplemented with glutamine (5 wt %).

At the end, the mice were subjected to social interaction and spatial memory tests. Cow's milk allergy is associated with disturbed social interaction and impaired spatial memory, and thus suited to study the effects of dietary intervention there.

### Social Interaction Tests

In the morning of the day after oral challenge mice were exposed to a social interaction test. Mice were placed in a 45×45 cm open field, with a small perforated Plexiglas cage located against one wall allowing visual, olfactory and minimal tactile interaction. During the habituation phase, mice were allowed to explore the room for 5 min. During the interaction phase, an age- and gender-matched unfamiliar target mouse was introduced in the cage for an additional 5 min. By using video tracking software (EthoVision 3.1.16, Noldus), an interaction zone around the cage was digitally determined. Time spent in the interaction zone as well as frequency of entering the interaction zone over the selected time period was measured.

### T Maze Spatial Memory Performance Test

T Maze Spontaneous Alternation is a behavioral test for measuring exploratory behavior and spatial memory performance in animals, especially rodent models for CNS disorders. This test is based on the willingness of rodents to explore a new environment, i.e. prefer to visit a new arm of the maze rather than a familiar arm. Many parts of the brain—including the hippocampus, septum, basal forebrain, and prefrontal cortex—are involved in this task.

Subjects are first placed in the start arm of the T Maze. Upon leaving the start arm, subjects choose between entering either the left or the right goal arm. With repeated trials, the animals should show less of a tendency to enter a previously visited arm. The percentage of alternation (number of turns in each goal arm) and total trial duration are recorded. This test is used to quantify cognitive deficits in transgenic strains of mice and evaluate novel chemical entities for their effects on cognition. After assessing social interaction on the day after the oral challenge, spontaneous alternation was tested in a T maze set-up. Each mouse was placed in the start arm and was allowed to explore the T maze until one of the goal arms was chosen. The animal was then returned to its home cage and performed a total of 5 trials. The T maze was thoroughly cleaned with 70% ethanol in between trials. The alternation ratio was defined as the amount of trials in which an animal alternated divided by the total amount of trials.

The results in terms of social interaction and T alternation were compared with the results observed for unsensitized mice not subjected to dietary intervention.

### Results

The cow's milk allergy model shows a good model for studying social interactions in a variety of ways. In table 1, the time in the interaction zone was significantly reduced for sensitized mice (control-sensitized mice vs. control-unsensitized mice). The effects of reduced social interaction induced by sensitization were diminished for all tested diets but greatly diminished for mice fed glutamine. Statistics on the in table 1 mentioned samples were generated using unpaired t-test vs CMA sensitized control to calculate p values. CMA for glutamine intervention was significantly improved over control CMA mice (p=0.09).

TABLE 1
Time spent in interaction zone (sec)

| | Control, | |
|---|---|---|
| | CMA Sensitized mice | CMA glutamine (‘Gln’) |
| Mean +/− s.e. | 75 +/− 12.1 (n = 12) | 117 +/− 20.8 (n = 12) |

Surprisingly, unsensitized mice that were fed the Gln diet, thus not investigated using the CMA model, showed a marked improvement in seeking longer periods of social interaction as measured by the time spent in the interaction zone over un-sensitized mice that were fed the control diet (141+/−23.6 sec vs 174+/−19.1 sec). No significant difference was observed in the distance moved for both sensitized and un-sensitized mice during the test phase. Distance moved is taken as an internal control to verify that the time spent in the interaction zone is not due to decreased movement of the mice.

The cow's milk allergy model also shows a good model for studying spatial memory using T-maze spontaneous alternation behavioral tests. In table 2, the effects of reduced alternation in the T-maze test induced by sensitization were markedly improved for mice fed a diet with supplementation of Gln), compared to the situation observed for control CMA-mice. Statistics on the in table 2 mentioned samples were generated using unpaired t-test vs CMA sensitized control to calculate p values.

TABLE 2
% Alternation in spatial memory test

| | Control, | |
|---|---|---|
| | CMA Sensitized mice | CMA Gln |
| Mean, s.e. | 52.7 +/− 4.3 (n = 9) | 66.7 +/− 6.4 (n = 12) |

The above tests clearly show an effect of glutamine intervention in terms of improved exploratory behavior, memory performance, particularly spatial memory performance, and social interaction both in non-allergic and in allergic mice, the latter suffering from impaired exploratory behavior, memory performance, particularly spatial memory performance, and social interaction.

### Example 2

### Preterm Formula with Glutamine

Preterm formula in powder form comprising per 100 g about 474 kcal, 15.6 g protein, 49.8 g digestible carbohydrates, 7.35 g of which is glucose, maltose and sucrose taken together, 22.9 g fat and 4.7 g non-digestible oligosaccharides. The protein comprises about 92.5 wt. % casein and whey protein from cow's milk based on total protein in a weight ratio of 1:1.5. About 7.5 wt. % of the protein is free L-glutamine. Fat is for the main part of vegetable origin but also tuna fish oil (source of DHA), algae oil (source of ARA) arachidonic acid (ARASCO, Martek) and egg lipid (source of DHA and ARA) are present as a source of LC-PUFA, resulting in 0.52 wt % ARA, 0.40 wt % DHA, and 0.08 wt % EPA based on total fatty acyl chains and capric acid, lauric acid, myristic acid and palmitic acid together representing a total of 12.4 wt % again based on total fatty acyl chains. Cytidine-, uridine- and adenosine-5′-monophosphate are present in a total amount of 3.3 mg per 100 kcal of the preterm formula. Furthermore the composition comprises minerals, trace elements, vitamins, and other micronutrients as known in the art and according to guidelines for preterm infants. For a ready to drink formula, the instructions are to dilute 16.9 g powder (3 scoops) with water until a final volume of 100 ml.

### Example 3

### Nutritional Supplement Enriched in Glutamine

Human milk fortifier in powder form packed in sachets comprising 2.1 g. Based on 100 g powder the composition comprises 25.2 g protein, 52.2 g digestible carbohydrates (4.3 g of which is glucose and maltose taken together), the rest being minerals, trace elements and vitamins. The protein comprises 50 wt % of whey protein hydrolysate and casein hydrolysate in a weight ratio of 1/1 and 50 wt % of free L-glutamine based on total protein. Also 10 g of a fat component rich in ARA, EPA and DHA is present. The nutritional supplement contains no non-digestible oligosaccharides.

### Example 4

### Kit for Preterms

A package with 20 sachets with powder comprising glutamine in free amino acid form, non-digestible oligosaccharides, maltodextrin. The non-digestible oligosaccharides are galacto-oligosaccharides (Source Vivinal GOS, Borculo Domo) and fructopolysaccharides (Source RaftilinHP, Orafti) in a weight ratio of 9:1. The weight ratio of glutamine to the sum of non-digestible oligosaccharides GOS and FOS is about 1:1.5. Maltodextrin is present in an amount of about 70 wt %. The package also comprises a second set of 20 sachets with 3×10⁹ cfu of Bifidobacterium breve M-16V (Morinaga) per g powder. The package is accompanied with instructions for use of the sachets for preterms, wherein the instructions are expressed in g/100 mL ready-to-feed product (with daily dosage of 150 ml for preterms): 0.2 g/100 mL glutamine and 0.4 g/100 mL GOS/FOS, and 1 g B. breve.

1-20. (canceled)

21. A method for improving exploratory behavior, memory performance and/or social interaction in an infant or toddler suffering from allergy or from intestinal inflammation or at risk of allergy; and/or treating or preventing impaired or decreased exploratory behavior, memory performance and/or social interaction in an infant or toddler, the method comprising administering to the infant glutamine or a nutritional composition comprising glutamine.

22. The method according to claim 21, wherein the infant or toddler at risk of allergy is a preterm infant, small for gestational age (SGA), or born via caesarian section.

23. The method according to claim 21, wherein the nutritional composition further comprises:
(a) one or more nucleotide(s) selected from the group consisting of cytidine 5′-monophosphate, uridine 5′-monophosphate, adenosine 5′-monophosphate, guanosine 5′-monophosphate and inosine 5′-monophosphate,
(b) a carbohydrate selected from the group consisting of glucose, maltose and sucrose, or a combination thereof, or
(c) a combination of (a) and (b).

24. The method according to claim 21, wherein the nutritional composition is substantially free of non-digestible oligosaccharides.

25. The method according to claim 21, wherein the memory performance comprises spatial memory performance or short-term or direct memory performance.

26. The method according to claim 21, wherein the composition does not comprise the combination of Bifidobacterium breve and a non-digestible oligosaccharide.

27. The method according to claim 21, wherein the nutritional composition further comprises arachidonic acid and/or docosahexaenoic acid.

28. The method according to claim 21, wherein the nutritional composition comprises protein, fat and/or digestible carbohydrates and is selected from the group consisting of an infant starter formula, an infant follow on formula, a toddler milk, a preterm formula, a post discharge formula and a human milk fortifier.

29. The method according to claim 21, wherein the glutamine is present in the form of free amino acids, dipeptides and/or tripeptides.

30. The method according to claim 21, wherein the composition comprises a total amount of the nucleotides (b) between 0.01 and 5 mg/100 kcal of the nutritional composition.

31. The method according to claim 21, wherein the total amount of carbohydrates (c) represents between 5 and 50% of total calories of the nutritional composition.

32. The method according to claim 21, wherein the total amount of carbohydrates (c) represents between 5 and 25% of total calories of the nutritional composition.

33. A method for providing nutrition to an infant or toddler suffering from allergy or from intestinal inflammation, or an infant or toddler at risk of allergy, the method comprising administering to the infant or toddler glutamine in the form of free amino acids and/or glutamine containing dipeptide and/or glutamine containing tripeptide, or a nutritional composition comprising glutamine in the form of free amino acids and/or glutamine containing dipeptide and/or glutamine containing tripeptide.

34. The method according to claim 33, wherein the infant or toddler at risk of allergy is a preterm infant, small for gestational age (SGA), or born via caesarian section.

35. The method according to claim 33, wherein the nutritional composition further comprises:
(a) one or more nucleotide(s) selected from the group consisting of cytidine 5′-monophosphate, uridine 5′-monophosphate, adenosine 5′-monophosphate, guanosine 5′-monophosphate and inosine 5′-monophosphate,
(b) a carbohydrate selected from the group consisting of glucose, maltose and sucrose, or a combination thereof, or
(c) a combination of (a) and (b).

36. A method for treating or preventing impaired or decreased exploratory behavior, memory performance and/or social interaction in an infant or toddler, comprising administering to the infant or toddler glutamine in the form of free amino acids and/or glutamine containing dipeptide and/or glutamine containing tripeptide, or a nutritional composition comprising glutamine in the form of free amino acids and/or glutamine containing dipeptide and/or glutamine containing tripeptide.

37. The method according to claim 36, wherein the nutritional composition further comprises:
(a) one or more nucleotide(s) selected from the group consisting of cytidine 5′-monophosphate, uridine 5′-monophosphate, adenosine 5′-monophosphate, guanosine 5′-monophosphate and inosine 5′-monophosphate,
(b) a carbohydrate selected from the group consisting of glucose, maltose and sucrose, or a combination thereof, or
(c) a combination of (a) and (b).

38. A kit of parts comprising a first container comprising glutamine, at least one non-digestible oligosaccharide selected from the group consisting fructo-oligosaccharides, galacto-oligosaccharides, gluco-oligosaccharides, arabino-oligosaccharides, mannan-oligosaccharides, xylo-oligosaccharides, fuco-oligosaccharides, arabinogalacto-oligosaccharides, glucomanno-oligosaccharides, galactomanno-oligosaccharides, sialic acid comprising oligosaccharides and uronic acid oligosaccharides and a carbohydrate selected from the group consisting of glucose, maltose, sucrose and maltodextrin, or a combination thereof; and a second container comprising Bifidobacterium breve.

39. The kit of parts according to claim 38, wherein the glutamine is in the form of free amino acids, dipeptides and/or tripeptides.

40. The kit of parts according to claim 38, wherein glutamine is present in the first container in an amount of between 1.5 and 20 wt. %, the non-digestible oligosaccharide is present in an amount of between 0.5 to 20 wt. %, and the carbohydrate is present in an amount of at least 50 wt. %.

41. The kit of parts according to claim 38, wherein fructo-oligosaccharides and galacto-oligosaccharides are present as non-digestible oligosaccharides, in a weight ratio of glutamine to the sum of fructo-oligosaccharides and galacto-oligosaccharides of between 2:1 and 1:4.

42. The kit of parts according to claim 41, wherein the weight ratio of glutamine to the sum of fructo-oligosaccharides and galacto-oligosaccharides is between 1:1 and 1:3.
